(19) United States
(12) Patent Application Publication
(10) Pub. No.: US 2010/0168584 A1
(43) Pub. Date: Jul. 1, 2010

(54) BIOLOGICAL OBSERVATION APPARATUS, BIOLOGICAL OBSERVATION METHOD, AND ENDOSCOPIC APPARATUS

(75) Inventors: Ken FUJINUMA, Tokyo (JP); Takeshi WATANABE, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/722,964

(22) Filed: Mar. 12, 2010

Related U.S. Application Data
(63) Continuation of application No. PCT/JP2008/071980, filed on Dec. 3, 2008.

(30) Foreign Application Priority Data
Dec. 25, 2007 (JP) 2007-333011

(51) Int. Cl. A61B 6/00

(52) U.S. Cl. 600/473

(57) ABSTRACT

A biological observation apparatus comprises: a light source unit capable of selectively emitting illumination light beams of at least two or more kinds of wavelength regions; an irradiation optical system for irradiating a subject with the illumination light beams; a detection optical system for detecting scattered light beams from the subject, and acquiring respective images with respect to the respective wavelength regions; and an image processing section for performing comparison operation processing on at least two or more kinds of acquired images, wherein said two or more kinds of wavelength regions are selected so that a ratio of at least one spectral characteristic coefficient between at least one biological tissue serving as an identification target and at least another one biological tissue differing from the identification target with respect to at least one wavelength region among said wavelength regions is different from a ratio of this spectral characteristic coefficient therebetween with respect to at least another one wavelength region differing from the concerned one wavelength region among said wavelength regions.

### TECHNICAL FIELD

The present invention relates to a biological observation apparatus, a biological observation method, and an endoscopic apparatus.

### BACKGROUND ART

Heretofore, there is a known imaging system for detecting the running pattern of blood vessels by irradiating a biological tissue with infrared light and capturing the image of the biological tissue (for example, refer to Patent Citation 1).

Patent Citation 1:

Japanese Unexamined Patent Application, Publication No. 2004-358051

### DISCLOSURE OF INVENTION

However, a surface structure of a biological tissue is much enhanced in an image observed by using conventional optical imaging method, because light is strongly scattered on a surface of biological tissue. Therefore, it makes difficult to observe a specific biological tissue serving as an identification target which is lying deep inside of the biological tissue. For example, fat tissue particularly scatters light on its surface, it is difficult to observe the running pattern of blood vessel which lying deep inside of fat tissue by using conventional optical imaging method.

The present invention was made to address such a situation, with an object of providing a biological observation apparatus and an endoscopic apparatus, with which the distribution statuses of two or more types of biological tissues such as fat and blood can be exclusively visualized by lessening the influence of the surface profile of a biological tissue.

In order to achieve the above object, the present invention provides the following solutions.

The present invention provides a biological observation apparatus comprising: a light source unit capable of selectively emitting illumination light beams of two or more kinds of wavelength regions which provide different spectral characteristic coefficients to two or more types of biological tissues; an irradiation optical system for irradiating a subject with the illumination light beams from the light source unit; a detection optical system for detecting scattered light beams when the subject is irradiated with the illumination light beams of the two or more kinds of wavelengths by the irradiation optical system, and acquiring respective images thereof; and an image processing section for performing comparison operation processing on two or more kinds of images acquired by the detection optical system.

According to the present invention, illumination light beams of two or more kinds of wavelengths are selectively emitted by the operation of the light source unit, and are irradiated on the subject by the irradiation optical system. The thus irradiated two or more kinds of illumination light beams are scattered by the subject, and are detected by the detection optical system, by which the respective images of these light beams are created.

Since the ratio of the spectral characteristic coefficient between the respective biological tissues differs per each illumination light beam, the ratio of data quantity between these biological tissues included in a scattered light beam resulting from the irradiation of an illumination light beam differs depending on each wavelength. Accordingly, by performing the comparison operation processing between these two or more kinds of images with the image processing section, it becomes possible to enhance two or more kinds of data of these two or more types of biological tissues, and conversely to reduce the data of the surface profile. Therefore, the distributions of two or more types of biological tissues deep inside a biological tissue can be visualized.

Moreover, in the present invention, as for the wavelength regions of the above-mentioned illumination light beams of two or more kinds of wavelengths, the wavelength regions are selected so that a scatter coefficient of respective biological tissue with respect to the at least one wavelength region is approximately equal to a scatter coefficient of the biological tissue with respect to the at least another one wavelength region differing from the concerned one wavelength region. If these scatter coefficients are approximately equal to each other, these light beams are spread to approximately same depths within a biological tissue. Accordingly, when the comparison operation processing between the two or more kinds of images is performed by the image processing section, the data of biological tissues at approximately same depths are subjected to the operation processing. Therefore, the distributions of two or more types of biological tissues deep inside a biological tissue can be accurately visualized.

In addition, as for a wavelength region of the above-mentioned illumination light beams of two or more kinds of wavelengths, the present invention uses a wavelength within an infrared region and/or a near-infrared region. Since biological tissues have smaller scatter coefficients as the wavelength gets longer, a penetration depth of a light with a longer wavelength becomes longer, thus the image acquired by using a light with a longer wavelength contains information of a deeper position within a biological tissue. Accordingly, the distributions of two or more types of biological tissues can be visualized in a deeper position within a biological tissue.

In this invention, the comparison operation processing may be a division operation which divides one of the images by another one of the images.

Moreover, in this invention, the comparison operation processing may be a subtraction operation which subtracts one of the images from another one of the images. At this time, the data of the surface profile can be more effectively reduced if the wavelength regions are selected so that a ratio of at least one spectral characteristic coefficient between at least one biological tissue serving as an identification target and at least another one biological tissue differing from the identification target with respect to at least one wavelength region among the two or more kinds of wavelength regions is approximately equal to 1.

In this invention, examples of the biological tissue can include a fat tissue, a subcutaneous tissue, a bone tissue, a muscular tissue, a skin tissue, blood, a blood vessel, a lymphatic fluid, a lymphatic vessel, a nerve tissue, a tumor tissue, a collagen, and a melanin.

The present invention provides a biological observation apparatus comprising: a light source unit for emitting an illumination light beam having two or more kinds or wavelength regions which provide different spectral characteristic coefficients to two or more types of biological tissues; an irradiation optical system for irradiating a subject with the illumination light beam from the light source unit; a spectral optical system for dividing a scattered light beam into scattered light beams of the two or more kinds of wavelengths, when the subject is irradiated with the illumination light beam having the two or more kinds of wavelengths by the irradiation optical system; and a detection optical system for respectively detecting the scattered light beams of the two or more kinds of wavelengths that have been divided by the spectral optical system, and acquiring images thereof; and an image processing section for performing comparison operation processing on two or more kinds of images acquired by the detection optical system.

Moreover, in this invention, if one of the biological tissues is blood, the wavelengths may be equal to or between 400 nm and 2300 nm.

Furthermore, in this invention, one of the wavelengths may be equal to or between 400 nm and 1100 nm or equal to or between 1400 nm and 2300 nm, and the other one of the wavelengths may be equal to or between 600 nm and 850 nm or equal to or between 1000 nm and 1400 nm.

In this way, it becomes possible to set the absorption coefficient of a substance contained in blood, for example a hemoglobin, to be 1 cm⁻¹ for the illumination light beam of one of the wavelengths and 5 cm⁻¹ for the illumination light beam of the other one of the wavelengths. Therefore, the running pattern of a blood vessel buried in the biological tissue, in particular a fat tissue, can be accurately visualized.

Moreover, in this invention, the one of the wavelengths may be equal to or between 400 nm and 600 nm or equal to or between 900 nm and 1100 nm.

In this way, it becomes possible to select a wavelength for which the absorption coefficient of a hemoglobin or water comes to a peak, as one of the wavelengths.

Furthermore, the present invention provides an endoscopic apparatus comprising any one of the above-mentioned biological observation apparatus.

The present invention offers an effect in which the distributions of at least two or more types of biological tissues deep inside an organism's body can be clearly detected despite the surface profile of the biological tissue.

### BRIEF DESCRIPTION OF DRAWINGS

FIG. 1A is an overall schematic block diagram showing a biological observation apparatus and an endoscopic apparatus according to a first embodiment of the present invention.

FIG. 1B is a front view of a filter turret of the endoscopic apparatus and the biological observation apparatus according to the first embodiment of the present invention.

FIG. 2A shows an image of a scattered light beam acquired when an illumination light beam of a first wavelength is irradiated by the endoscopic apparatus of FIG. 1A.

FIG. 2B shows the optical path when the illumination light beam of the first wavelength is irradiated by the endoscopic apparatus of FIG. 1A.

FIG. 3A shows an image of a scattered light beam acquired when an illumination light beam of a second wavelength is irradiated by the endoscopic apparatus of FIG. 1A.

FIG. 3B shows the optical path when the illumination light beam of the second wavelength is irradiated by the endoscopic apparatus of FIG. 1A.

FIG. 4 shows an example of an image created by comparison operation processing with the image processing section of the endoscopic apparatus of FIG. 1A.

FIG. 5 is an overall schematic block diagram showing a first modified example of the biological observation apparatus and the endoscopic apparatus of FIG. 1A.

FIG. 6 is an overall schematic block diagram showing a second modified example of the biological observation apparatus and the endoscopic apparatus of FIG. 1A.

FIG. 7 is an overall schematic block diagram showing a biological observation apparatus and an endoscopic apparatus according to a second embodiment of the present invention.

FIG. 8 is an overall schematic block diagram showing a modified example of the biological observation apparatus and the endoscopic apparatus of FIG. 7.

### EXPLANATION OF REFERENCE

A: Biological tissue (subject)
λ1 and λ2: Wavelength
G1 and G2: Image
L1 and L2: Illumination light beam
S1 and S2: Scattered light beam
1: Biological observation apparatus
2: Endoscopic apparatus
4: Light source device (light source unit)
7: Light guide (irradiation optical system)
8: Object lens (detection optical system)
9: Image guide (detection optical system)
12, 12a, and 12b: Imaging section (detection optical system)
14: Image processing section
16: Spectral section (spectral optical system, light source switchover circuit)

### BEST MODE FOR CARRYING OUT THE INVENTION

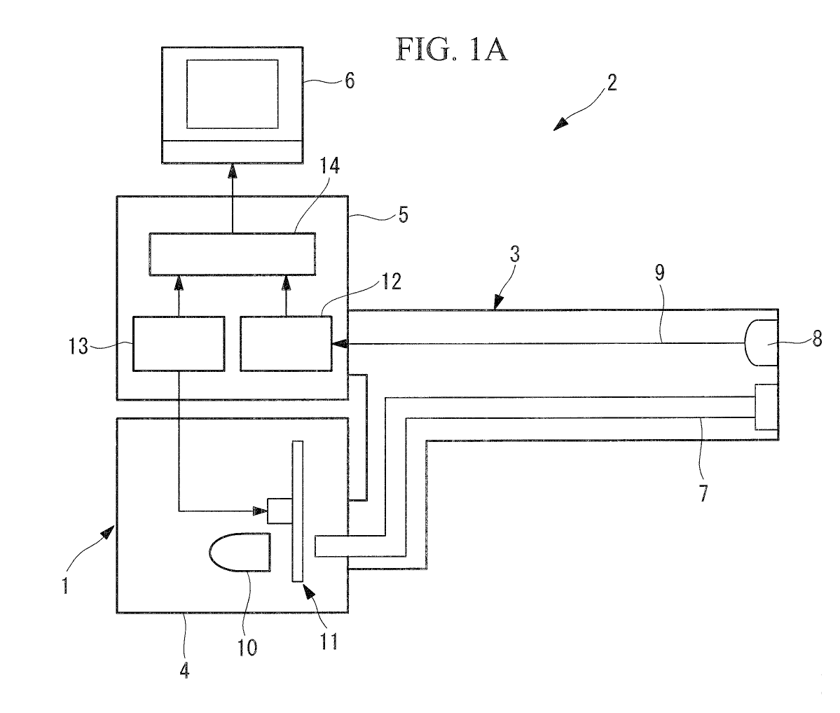

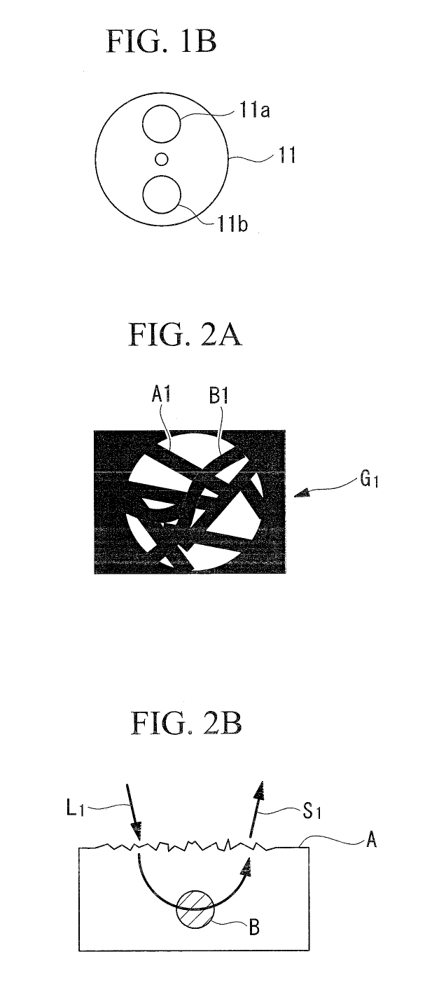

Hereunder is a description of a biological observation apparatus 1 and an endoscopic apparatus 2 according to a first embodiment of the present invention, with reference to FIG. 1A through FIG. 4.

The biological observation apparatus 1 of this embodiment is equipped in the endoscopic apparatus 2 which has a structure for visualizing the distributions of two types of biological tissues deep inside an organism's body.

As shown in FIG. 1A, the endoscopic apparatus 2 comprises: a long and slender insertion portion 3 to be inserted in a body cavity; a light source device (light source unit) 4 placed on the proximal side of the insertion portion 3; a camera control unit (CCU) 5 for detecting a light beam which has been condensed at the distal end of the insertion portion 3, making an image of it, and applying an image processing thereto; and a monitor 6 for displaying the image that has been image-processed by the CCU 5.

The insertion portion 3 comprises: a light guide (irradiation optical system) 7 for guiding a light beam emitting from the light source device 4, along the longitudinal direction from the proximal side to the distal end of the insertion portion 3, to thereby irradiate an inner surface of a body cavity with the light beam; an object lens 8 for condensing a scattered light beam which has traveled from the inner surface of the body cavity into the inside thereof, then has been scattered by the biological tissue and the like, and now is returning to the inner surface of the body cavity; and an image guide 9 for guiding the scattered light beam that has been condensed by the object lens 8.

The light source device 4 comprises: a light source 10 such as a xenon lamp, a halogen lamp, a white LED, and a near-infrared LED, for generating a light beam of a relatively broad wavelength band including two kinds of wavelengths; and a filter turret 11 as a wavelength-switchover means, for selectively taking out light beams having two kinds of wavelengths from the light beam which has been emitted from the light source 10. As shown in FIG. 1B, the filter turret 11 comprises two filters 11a and 11b that respectively allow the transmissions of light beams having two kinds of wavelengths a first wavelength λ1 and a second wavelength λ2, corresponding to the biological substance. Here, as for the wavelength-selection means other than the filter turret 11, it is possible to use a spectral element which diffracts a light beam by grating, or it is also possible to execute video-rate imaging by a driven-type spectral device that is capable of a high-speed switchover operation between wavelengths (for example, a Fabry-Perot type wavelength variable element and a piezo-driven etalon spectral element).

The CCU 5 comprises: an imaging section 12 such as a CCD for capturing the scattered light beam that has been guided through the image guide 9; a control circuit 13 for associating the timing for switching between the respective filters 11a and 11b of the filter turret 11, with the image acquired by the imaging section 12; and an image processing section 14 for processing the acquired image.

The image processing section 14 creates a new image by comparison operation processing (for example, subtraction and/or division) the image acquired when the illumination light beam of the second wavelength λ2 is irradiated from the image acquired when the illumination light beam of the first wavelength λ1 is irradiated.

The biological observation apparatus 1 of this embodiment comprises the light source device 4, the light guide 7, the object lens 8, the image guide 9, the imaging section 12, the control circuit 13, and the image processing section 14.

Here is a description of the operation of the thus configured biological observation apparatus 1 and the thus configured endoscopic apparatus 2 according to this embodiment.

For the observation of an inner wall of a body cavity with the endoscopic apparatus 2 of this embodiment, the light source 10 of the light source device 4 is turned on to emit an illumination light beam, and the control circuit 13 controls the filter turret 11 to rotate so that the different filters 11a and 11b can be alternately placed on the optical axis of the illumination light beam. In this way, the illumination light beam of the first wavelength λ1 and the illumination light beam of the second wavelength λ2 are alternately emitted from the light source device 4. Preferably, the control circuit 13 controls the filters 11a and 11b to pass by or stop at the optical axis of the illumination light beam, so that they can be repeatedly placed on the axis, so as to thereby carry out the observation by each wavelength in a timewise manner according to the movement inside the organism's body.

The two kinds of the illumination light beams alternately emitting from the light source device 4 are respectively guided through the light guide 7 of the insertion portion 3 to the distal end thereof, and then emit from the distal end of the insertion portion 3 toward the inner wall of the body cavity.

At this time, if the selection is made so that the ratio of the absorption coefficient between the biological tissue A and the biological tissue B (absorption coefficient of biological tissue B/absorption coefficient of biological tissue A) with respect to the illumination light beam L1 of the first wavelength λ1 exceeds 1, a light beam passing through the biological tissue B that lies deep inside the biological tissue A, is absorbed by the biological tissue B and thus is scattered. Therefore, as shown in FIG. 2A, the scattered light beam S1 which is again emitting from the surface of the biological tissue A will include both the data B1 of the biological tissue B and the data A1 of the surface profile of the biological tissue A.

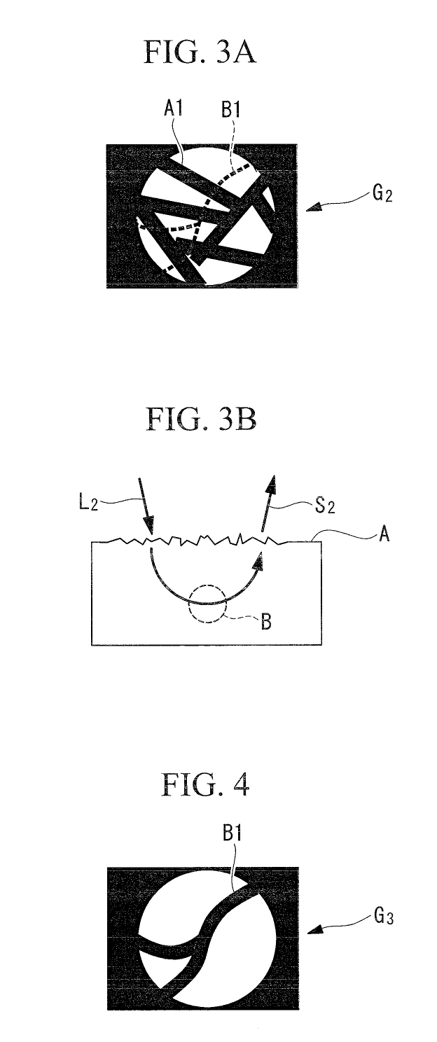

On the other hand, as shown in FIG. 3B, if the selection is made so that the ratio of the absorption coefficient between the biological tissue A and the biological tissue B (absorption coefficient of biological tissue B/absorption coefficient of biological tissue A) with respect to the illumination light beam L2 of the second wavelength λ2 is lower than that of the illumination light beam L1, the proportion of the illumination light beam L2 to be absorbed by the biological tissue B will be smaller than that of the illumination light beam L1. Therefore, as shown in FIG. 3A, the scattered light beam S2 which has once entered from the inner wall of the body cavity into the biological tissue A and is again emitting from the surface of the biological tissue A will include less information B1 of the biological tissue B (broken line) but abundantly include the information A1 of the surface profile of the biological tissue A.

Then, the scattered light beams S1 and S2 resulting from the irradiation of these two kinds of illumination light beams L1 and L2 are respectively captured and made into images by the imaging section 12, and thereafter the respective images G1 and G2 are subjected to a classification regarding which kind of the illumination light beam L1 or L2 made the image G1 or G2, by the operation of the control circuit 13. In the image processing section 14, the image G1 resulting from the irradiation of the illumination light beam L1 of the first wavelength λ1 is subtracted by the image G2 resulting from the irradiation of the illumination light beam L2 of the second wavelength λ2. In the thus obtained differential image G3, as shown in FIG. 4, only the image of the data B1 of the blood vessel B appears as the difference, which is to be displayed on the monitor 6.

The comparison operation processing is, for example, subtraction and/or division, or combination thereof.

At this time, if the selection of λ1 and λ2 is made so that the illumination light beam L1 can make a significant difference in the absorption coefficient of at least 1 cm⁻¹ or greater between these biological tissues, and the illumination light beam L2 can provide equivalent absorption coefficients to these biological tissues, it will become possible to create an image in which the data A1 of the biological tissue A has been removed or sufficiently reduced by performing the comparison operation between images acquired with the illumination light beams L1 and L2. Accordingly, the biological observation apparatus 1 and the endoscopic apparatus 2 of this embodiment are advantageously capable of visualizing the biological tissue B which lies deep inside the biological tissue A more clearly despite the existence of the biological tissue A.

Once the running pattern of the biological tissue B which lies deep inside the biological tissue A is visualized, the risk of injuring the biological tissue B can be readily avoided at the time for incising a human body, and therefore the operation time can be shortened.

In particular, since the light wavelength-dependent characteristic of the biological tissue A is utilized to visualize the distribution of the biological tissue B, this method is noninvasive and capable of alleviating the burden on the patient, and enables an inexpensive observation without using CT and MRI.

In this embodiment, in the case where the biological tissue B is the blood vessel, a wavelength meeting a range of 400 nm≦λ1≦600 nm may be selected for the first wavelength λ1, and a wavelength meeting a range of 1000 nm≦λ2≦1400 nm is selected for the second wavelength λ2. It is also possible to use a wavelength meeting a range of 400 nm≦λ1≦1100 or 1400 nm≦λ1≦2300 nm as the first wavelength λ1, and to use a wavelength meeting a range of 600 nm≦λ2≦850 nm or 1000 nm≦λ2≦1400 nm as the second wavelength λ2.

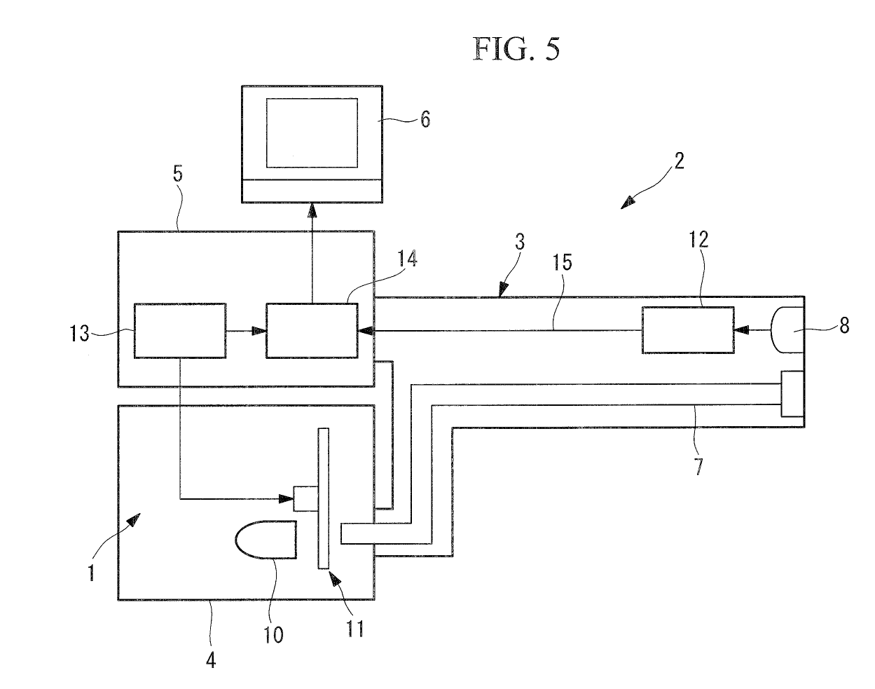

In addition, in this embodiment, the imaging section 12 such as a CCD is set in the CCU 5 that is provided on the proximal side of the insertion portion 3. However, instead of this arrangement, it is also possible as shown in FIG. 5 to set the imaging section 12 on the distal end of the insertion portion 3 so that an electric signal converted from the image data can be transmitted to the CCU 5 through a wire 15.

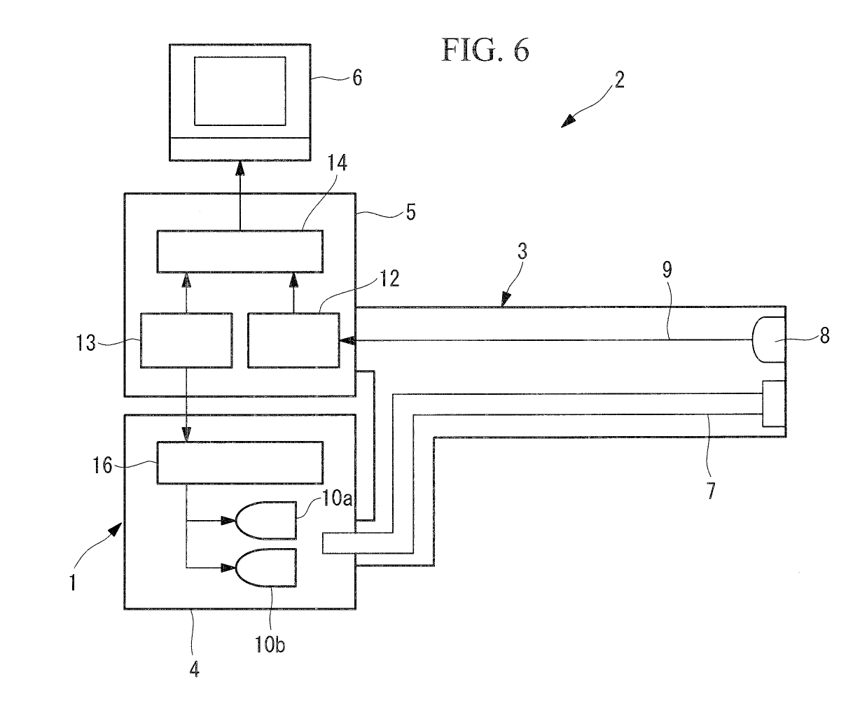

Moreover, in this embodiment, the wavelengths λ1 and λ2 of the illumination light beams L1 and L2 emitting from the light source device 4 are switched over by the rotation of the filter turret 11. However, instead of this configuration, it is also possible as shown in FIG. 6 to separately prepare light sources 10a and 10b whose center wavelengths are respectively the first wavelength λ1 and the second wavelength λ2 so that the lighting of the light source 10a of the first wavelength λ1 and the lighting of the light source 10b of the second wavelength λ2 can be alternately switched over according to the command signal from a light source switchover circuit 16.

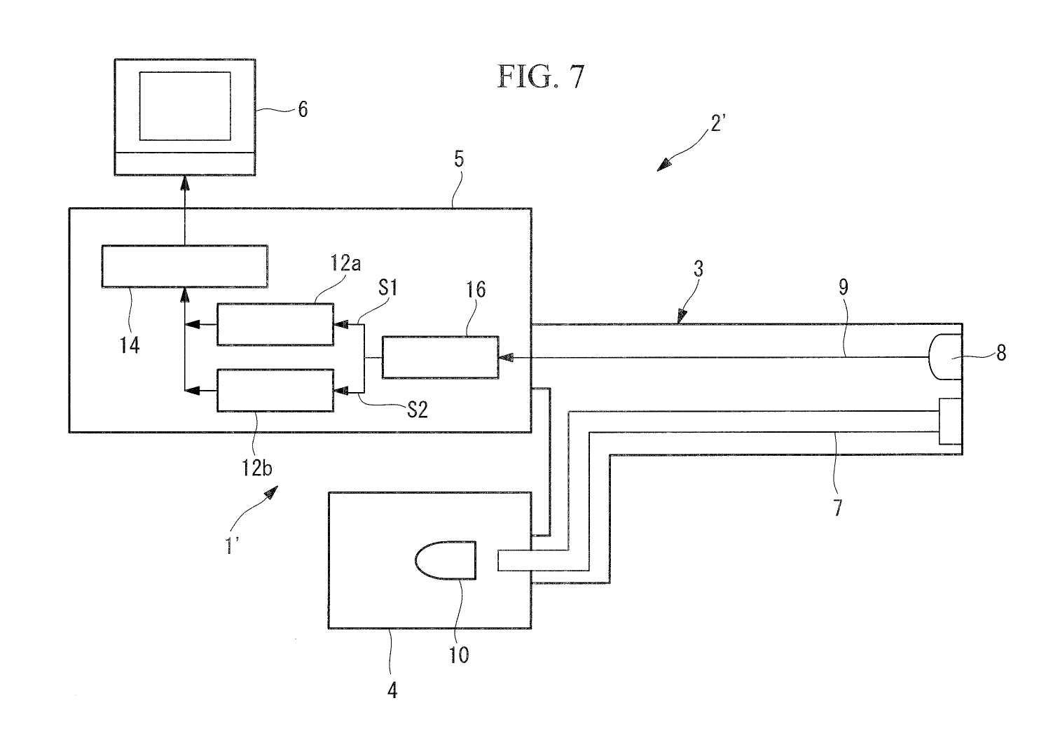

Next, hereunder is a description of a biological observation apparatus 1′ and an endoscopic apparatus 2′ according to a second embodiment of the present invention, with reference to FIG. 7.

In the description of this embodiment, parts sharing common structures with those of the biological observation apparatus 1 and the endoscopic apparatus 2 of the above-mentioned first embodiment are denoted by the same reference symbols, and are not explained herein.

The biological observation apparatus 1° of this embodiment is different from the biological observation apparatus 1 of the first embodiment, in the point where the filter turret 11 is not provided in the light source device 4, but instead a spectral section 16 for dividing the scattered light beam that has been guided through the image guide 9 into two beams is provided in the CCU 5. In addition, the biological observation apparatus 1′ of this embodiment is different from the biological observation apparatus 1 of the first embodiment, also in the point where two imaging sections 12a and 12b are provided so as to respectively capture the thus divided two scattered light beams.

Examples of the spectral section 16 can include a dichroic mirror, a diffraction grating, and a prism. The spectral section 16 is designed to respectively extract the scattered light beams S1 and S2 having the wavelengths λ1 and λ2 by allowing the transmission of the scattered light beam that has been guided through the image guide 9. Then, the scattered light beam S1 of the first wavelength λ1 is captured by the first imaging section 12a, and the scattered light beam S2 of the second wavelength λ2 is captured by the second imaging section 12b.

The scattered light beam S1 of the first wavelength λ1 extracted from the scattered light beam includes both the data B1 of the biological tissue B and the data A1 of the surface profile of the biological tissue A, while the scattered light beam S2 of the second wavelength λ2 extracted from the scattered light beam abundantly includes data A1 of the surface profile of the biological tissue A and includes less data B1 of the biological tissue B (dotted line).

Accordingly, by performing the comparison operation processing between the images G1 and G2 which have been respectively acquired by capturing the thus divided scattered light beams S1 and S2, it becomes possible to create the image G3 in which the data A1 of the surface profile of the biological tissue A has been removed and the data B1 of the biological tissue B clearly comes out.

In this embodiment, the spectral section 16 and the two imaging sections 12a and 12b are set in the CCU 5. However, instead of this setting, it is also possible to set them at the distal end of the insertion portion 3. In this case, electric signals output from the respective imaging sections 12a and 12b may be transmitted to the image processing section 14 of the CCU 5.

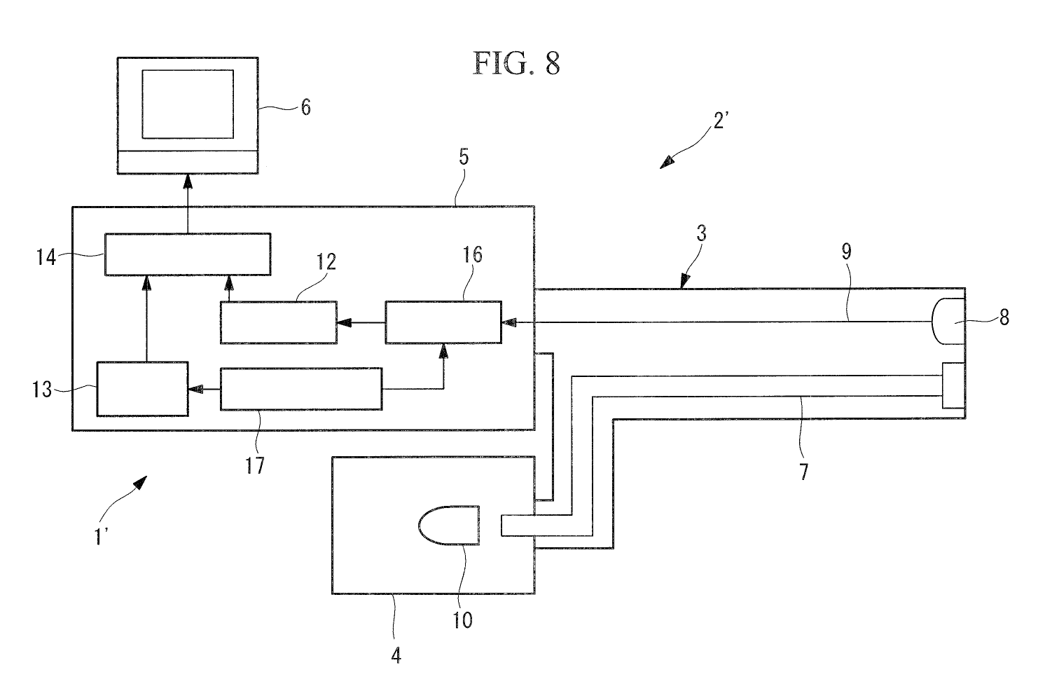

In addition, in this embodiment, as for the spectral section 16, it is also possible to employ a device as shown in FIG. 8 which can switch over the wavelength of the scattered light beam passing therethrough according to the command signal from a spectral switchover section 17. In this way, only one imaging section 12 is needed, and therefore the size and the cost can be much more reduced. In this case, the control circuit 13 can be used for associating the switchover timing for the spectral switchover section 17 with the acquired image G1 or G2.

The present invention is not to be limited to the above-mentioned embodiments, and various modifications can be made on the basis of the aforementioned gist. For example, the biological substances serving as the observation target of the present invention are a fat tissue, a subcutaneous tissue, a bone tissue, a muscular tissue, a dermal tissue, blood, a blood vessel, a lymphatic vessel, a lymphatic fluid, a lymph node, a nerve cell, a tumor cell, a collagen, and a melanin. In this embodiment, the biological tissue A is, for example, a fat tissue, a subcutaneous tissue, a bone tissue, a muscular tissue, or a dermal tissue. The biological tissue B is, for example, blood, a blood vessel, a lymphatic vessel, a lymphatic fluid, a lymph node, a nerve cell, a tumor cell, a collagen, and a melanin. After all, the present invention can be applicable because the observation of any substance having a distinct characteristic of light absorption in an organism's body is enabled by minimizing the influence of another intervening substance. In addition, it is preferable to modify the wavelength range of the illumination light beam to be used, according to the target biological substance. Moreover, it is also possible to modify the wavelength of the illumination light beam to be used, according to other biological substances. If three or more types of biological substances are mixedly present or overlaid deep inside an organism's body, it is also possible to alternately irradiate light beams having three kinds of wavelengths or more to perform the comparison operation processing on the respective images thereof.

Moreover, in this embodiment, an enhanced image of the biological tissue B is displayed upon the completion of the comparison operation processing. However, if conversely an image exclusively showing an enhanced image of the biological tissue A in which the data B1 of the biological tissue B has been completely removed by extracting an image data being cut a graphic image being recognized of the enhanced image of the biological tissue A in the image G1, or a composite image made of respectively enhanced images of both of the biological tissues A and B is displayed on the monitor, it will also become possible to even more contribute to endoscopic treatments (for example, fat tissue resection).

Furthermore, real time observation of an image which follows the movement of the organism's body per se or the biological substance, or an image which reflects the latest concentration or density of the biological substance, is also possible by acquiring image-processed images in a timewise manner and displaying them on the monitor. In addition, the applicable endoscope of the present invention is not limited to the above-mentioned embodiments, and may be various types of endoscopes (such as a rigid endoscope, a flexible endoscope, an endoscope for laparoscopic surgery, and a capsule endoscope), and a device or a system being an ensemble of a treatment instrument for surgical procedures such as grasping, resection, and suturing, and an endoscope which has another observation means such as fluorescence, polarized light, or ultrasonic waves.

1. A biological observation apparatus comprising:
a light source unit capable of selectively emitting illumination light beams of at least two or more kinds of wavelength regions;
an irradiation optical system for irradiating a subject with the illumination light beams;
a detection optical system for detecting scattered light beams from the subject, and acquiring respective images with respect to the respective wavelength regions; and
an image processing section for performing comparison operation processing on at least two or more kinds of acquired images, wherein
said two or more kinds of wavelength regions are selected so that a ratio of at least one spectral characteristic coefficient between at least one biological tissue serving as an identification target and at least another one biological tissue differing from the identification target with respect to at least one wavelength region among said wavelength regions is different from a ratio of this spectral characteristic coefficient therebetween with respect to at least another one wavelength region differing from the concerned one wavelength region among said wavelength regions.

2. A biological observation apparatus according to claim 1, wherein a scatter coefficient of said biological tissue with respect to the at least one wavelength region among said wavelength regions is approximately equal to a scatter coefficient of the biological tissue with respect to the at least another one wavelength region differing from the concerned one wavelength region among said wavelength regions.

3. A biological observation apparatus according to claim 1, wherein at least one wavelength region among said two or more kinds of wavelength regions is within an infrared region and/or a near-infrared region.

4. A biological observation apparatus according to claim 1, wherein said spectral characteristic coefficient is an absorption coefficient and/or a scatter coefficient.

5. A biological observation apparatus according to claim 1, wherein said comparison operation processing is a division operation.

6. A biological observation apparatus according to claim 1, wherein said comparison operation processing is a subtraction operation.

7. A biological observation apparatus according to claim 6, wherein a ratio of at least one spectral characteristic coefficient between at least one biological tissue serving as an identification target and at least another one biological tissue differing from the identification target with respect to at least one wavelength region among said two or more kinds of wavelength regions is approximately equal to 1.

8. An endoscopic apparatus comprising the biological observation apparatus according to claim 1.

9. A biological observation method comprising:
a light source capable of selectively emitting illumination light beams of at least two or more kinds of wavelength regions;
an irradiation step of irradiating a subject with the illumination light beams;
a detection step of detecting scattered light beams from the subject, and acquiring respective images with respect to the respective wavelength regions; and
an image processing step of performing comparison operation processing on at least two or more kinds of acquired images, wherein
said two or more kinds of wavelength regions are selected so that a ratio of at least one spectral characteristic coefficient between at least one biological tissue serving as an identification target and at least another one biological tissue differing from the identification target with respect to at least one wavelength region among said wavelength regions is different from a ratio of this spectral characteristic coefficient therebetween with respect to at least another one wavelength region differing from the concerned one wavelength region among said wavelength regions.

10. A biological observation method according to claim 9, wherein a scatter coefficient of said biological tissue with respect to the at least one wavelength region among said wavelength regions is approximately equal to a scatter coefficient of the biological tissue with respect to the at least another one wavelength region differing from the concerned one wavelength region among said wavelength regions.
